(19) United States
(12) Patent Application Publication
(10) Pub. No.: US 2020/0199513 A1
(43) Pub. Date: Jun. 25, 2020

(54) SYSTEM AND A METHOD FOR PRODUCING AN ENCAPSULATED CELLULAR SPHEROID

(71) Applicant: Arctoris Limited, Oxford (GB)

(72) Inventor: Alice Poppy Roworth, Oxford (GB)

(21) Appl. No.: 16/719,417

(22) Filed: Dec. 18, 2019

Related U.S. Application Data
(60) Provisional application No. 62/782,138, filed on Dec. 19, 2018.

(51) Int. Cl. C12M 1/12; C12N 5/071; C12M 3/06

(52) U.S. Cl. CPC C12M 25/16; C12N 2513/00; C12M 23/16; C12N 5/0602

(57) ABSTRACT

A system for producing an encapsulated cellular spheroid is disclosed. The system includes a source liquid reservoir containing a source liquid including a mixture of a living cell suspension and a liquid extracellular analog; a vertical solidification column containing a carrier liquid having a greater density than a density of the source liquid; and a source liquid injector in fluid communication with the source liquid reservoir and the vertical solidification column and being arranged to dispense droplets of the source liquid into a lower portion of the vertical solidification column, and heat to a temperature greater than a threshold temperature of the extracellular analog, wherein the liquid extracellular analog of the source liquid droplets irreversibly semi-solidifies at the temperature and encapsulates the living cell suspension within, such that living cells in the encapsulated living cell suspension adhere to one another in a spherical mass and form the encapsulated cellular spheroid.

### CROSS-REFERENCE TO RELATED APPLICATION

This application claims priority to U.S. Provisional Application No. 62/782,138, filed Dec. 19, 2018 and entitled, “System and a Method for Producing a Semisolid Sphere of an Extracellular Analog and an Encapsulated Cellular Spheroid,” which application is hereby incorporated by reference in its entirety in this application.

### TECHNOLOGICAL FIELD

The present disclosure relates generally to cell culturing and in particular to a system and method for producing an encapsulated cellular spheroid.

### BACKGROUND

Research seeking to understand the nature of cancer and to cure or prevent cancer is conducted using living cancer cells and various reagents that affect the growth and propagation of cancer cells and others used to indicate or measure those effects.

Typically, in-vitro (occurring or made to occur in a laboratory vessel or other controlled experimental environment rather than within a living organism or natural setting) cancer research experiments are conducted using cancer cells infused within a nutrient solution contained within flasks or well plates (a rectilinear device, generally of an industry standard size, comprising an array of compartments where a living cell suspension may be dispensed together with other liquid substances, to be examined later, in order to determine the effect of the other liquid substances on the living cells), where the cancer cells grow as a two-dimensional monolayer of cells across the bottom of the container. However, this environment does not compare favorably to the actual environment where cancer cells live and grow.

In an object such as the human body (in-vivo), cancer cells generally live and grow in a three-dimensional environment comprising living cells (both cancerous and non-cancerous) within an extracellular matrix comprised of collagens (any of a class of extracellular proteins abundant in higher animals, especially in the skin, bone, cartilage, tendon, and teeth, forming strong insoluble fibers and serving as connective tissue between cells, yielding gelatin when denatured by boiling) and related organic substances.

Current in-vitro cancer research may yield methods that appear to be effective in the laboratory, but often prove to be ineffective when translated to in-vivo environments or to living patients. At the same time, effective methods may fail to be discovered because laboratory research does not sufficiently replicate the in-vivo environment of the human body. Three-dimensional cancer-cell spheroids have been used to bridge the gap between in-vitro and in-vivo models and have shown to mimic an in-vivo response to both radiotherapy and drugs. By using three-dimensional environments for cancer research, it is hoped that unsuccessful, yet costly and time-consuming, in-vivo studies will be reduced by eliminating poor treatment candidates before they reach the in-vivo stage.

It has been observed that cancer cells often form spherical colonies within the interstitial extracellular environment of living organisms, instead of growing randomly. Based upon this observation, there is growing consensus that cancer cell spheroids are an important model for cancer treatment research. Importantly, cancer spheroids have shown more similar response to drug or radiation treatments than traditional two-dimensional in-vitro models.

Cancer research utilizing three-dimensional cancer-cell spheroids is dependent on effective methods for cultivating these cell colonies in a form that is compatible with and can be used in current laboratory procedures. Typically, to induce formation of a three-dimensional cancer cell spheroid (hereinafter referred to as a cellular spheroid), a vessel with a rounded bottom (similar in cross-sectional profile to the bottom of the letter U), coated to inhibit cell adherence and two-dimensional colony formation, is used to culture the cells of interest, where the rounded bottom serves to gather the cells together. This is currently done using laboratory well plates comprising low-attachment material where each well has a rounded bottom. Alternatively, this may be done using conventional laboratory well plates with flat bottoms where cellular spheroid formation may be induced by adding a concave (curved like a segment of the interior of a circle or hollow sphere) layer of agarose (a gel-like substance, which the cells generally do not attach to, obtained from agar used, in the context of cancer cell research, as a substrate for coating microplates in order to cultivate cancer cell spheroids) to each well before dispensing the cell suspension (living cells dispersed within a nutrient media by mixing or agitation).

As another alternative, formation of a three dimensional cellular spheroid may be induced by dispensing a cell suspension, as a number of individual drops, on a hydrophobic surface such as the surface of an inverted petri dish lid, whereby each drop becomes an environment with a non-adherent rounded bottom when the petri dish lid is reverted to its proper orientation and placed on the lower portion of the petri dish. As yet another alternative, rather than relying on the shape of the cell culture environment, cells may be injected directly into a semi-solid extracellular analog gel where the injected cells will be surrounded by the gel thereby inducing formation of a cellular spheroid. Other methods have been and continue to be developed including, for example, use of microfluidic devices.

While some cancer cells form cellular spheroids in experimental containers without an extracellular matrix, others require some form of extracellular analog for viability and correct cellular spheroid formation in an in-vitro environment. In such cases, to better mimic in-vivo environments, collagen, laminins, or other matrix proteins are added to the experimental cell suspension (both at a temperature less than a liquid-solid threshold (a temperature where an extracellular analog will remain liquid so long as its temperature remains below said temperature and will irreversibly solidify when the its temperature rises above said temperature) of collagen, the collagen plus cell suspension mixture is dispensed into the experimental container and then warmed to, for example, body temperature (i.e., 37 degrees Celsius (° C.)) whereupon the collagen polymerizes to become gel-like and forms a semi-solid extracellular analog that supports cell growth and, in the right circumstances, formation of cellular spheroids within the scaffolding provided by the matrix protein.

Today, preparation of cellular spheroids using an extracellular analog is laborious. A scientist may use specially coated low-attachment, round bottom well plates (although these are often not effective at preventing monolayer growth and promoting cellular spheroid growth) or they may use conventional flat bottom well plates by preparing a concave layer of agarose in each well, preparing a collagen (or other commercially available extracellular matrix mix) plus cell suspension mixture, and manually dispensing the mixture into the wells of the well plate.

To complicate matters, liquid collagen, and similar materials, must be kept below 4° C. until dispensed into the well plate, otherwise it solidifies and cannot be used. The day after dispensing the collagen plus cell suspension mixture the scientist must add a layer of warm nutrient media over the solidified collagen to prevent drying and provide a good gas-exchange interface. Then, the cells must incubate for a few days before it can be determined if they have formed cellular spheroids and if the cellular spheroids are of a consistent size and architecture. Moreover, the cultivation environment comprising a well, a collagen plus cell suspension mixture dispensed into the well, solidified with nutrient media dispensed over the top, with gaseous exchange only at the top of the collagen plus cell suspension mixture, is asymmetric wherein spheroid formation may tend to be asymmetric rather than resulting in substantially symmetric form. Because of these and other difficulties, only specialized laboratories utilize cancer cell spheroids in their routine research despite the obvious benefits in cancer research and drug discovery.

Accordingly, a need exists for a method and system for producing an encapsulated cellular spheroid that overcomes the issues noted hereinabove.

### BRIEF SUMMARY

The present disclosure provides a system and a method for producing an encapsulated cellular spheroid. The present disclosure includes, without limitation, the following example implementations.

Some example implementations provide an encapsulated cellular spheroid comprises a spherical mass of living cells adhering to one another and living within a matrix formed by an extracellular analog; the extracellular analog infused within and substantially surrounding the spherical mass of living cells and irreversibly semi-solidifying at a temperature greater than a liquid-solid threshold temperature of the extracellular analog so as to encapsulate the spherical mass of living cells within and form the encapsulated cellular spheroid.

In some example implementations of the spheroid of any preceding example implementation, or any combination of any preceding example implementations, the extracellular analog is collagen.

In some example implementations of the spheroid of any preceding example implementation, or any combination of any preceding example implementations, the living cells are cancer cells.

Some example implementations provide a system for producing an encapsulated cellular spheroid comprises a source liquid reservoir containing a source liquid comprising a mixture of a living cell suspension and a liquid extracellular analog at a temperature less than a threshold temperature of the extracellular analog; a vertical solidification column containing a carrier liquid at a temperature greater than the threshold temperature of the extracellular analog and having a greater density than a density of the source liquid; and a source liquid injector in fluid communication with the source liquid reservoir and the vertical solidification column and being arranged to dispense droplets of the source liquid into a lower portion of the vertical solidification column, the greater density of the carrier liquid allowing the lesser dense source liquid droplets to travel from the lower portion of the vertical solidification column to an upper portion thereof and heat to the temperature greater than the threshold temperature of the extracellular analog, wherein the liquid extracellular analog of the source liquid droplets irreversibly semi-solidifies at the temperature and encapsulates the living cell suspension within, such that living cells in the encapsulated living cell suspension adhere to one another in a spherical mass and form the encapsulated cellular spheroid.

In some example implementations of the system of any preceding example implementation, or any combination of any preceding example implementations, the vertical solidification column defines a height extending between the lower portion and the upper portion which corresponds to an amount of time that it takes for the dispensed source liquid droplets to heat to the temperature greater than the liquid-solid threshold temperature.

In some example implementations of the system of any preceding example implementation, or any combination of any preceding example implementations, the source liquid injector includes an aperture through which the source liquid droplets are dispensed into the carrier liquid contained in the vertical solidification column, and wherein a volume of each of the dispensed source liquid droplets is determined by a size of the aperture, a flow rate of the source liquid through the source liquid injector, a surface tension of the source liquid, and a buoyancy of the source liquid droplets within the carrier fluid.

In some example implementations of the system of any preceding example implementation, or any combination of any preceding example implementations, the system further comprises a source liquid stirring mechanism arranged to stir the source liquid contained in the source liquid reservoir; a source liquid cooler arranged to maintain a temperature of the source liquid contained in the source liquid reservoir at a temperature less than the liquid-solid threshold temperature of the extracellular analog; and a source liquid pump in fluid communication with the source liquid reservoir that controls a flow rate of the source liquid and directs the source liquid to the source liquid injector.

In some example implementations of the system of any preceding example implementation, or any combination of any preceding example implementations, the system further comprises a flow guide in fluid communication with the vertical solidification column and arranged to direct the encapsulated cellular spheroid from the upper portion of the vertical solidification column into a flow of circulating carrier liquid.

In some example implementations of the system of any preceding example implementation, or any combination of any preceding example implementations, the system further comprises a spheroid separator arranged after the flow guide and along a direction of the flow of circulating carrier liquid, and configured to separate the encapsulated cellular spheroid from the flow of circulating carrier liquid and direct the encapsulated cellular spheroid to a spheroid collector and direct the flow of circulating carrier liquid to a carrier liquid reservoir in fluid communication with the vertical solidification column.

In some example implementations of the system of any preceding example implementation, or any combination of any preceding example implementations, the system further comprises a carrier liquid filter arranged to receive the flow of circulating carrier liquid from the spheroid separator, remove residual semi-solid source liquid from the flow of circulating carrier liquid, and direct the flow of circulating carrier liquid to the carrier liquid reservoir; a carrier liquid heater arranged to maintain the circulating carrier liquid contained in the carrier liquid reservoir at a temperature greater than the liquid-solid threshold of the extracellular analog; and a carrier liquid circulating pump configured to maintain the flow of circulating carrier liquid through the vertical solidification column.

In some example implementations of the system of any preceding example implementation, or any combination of any preceding example implementations, the system further comprises a container of nutrient media arranged to receive one or more of the encapsulated cellular spheroids separated by the spheroid separator from the flow of circulating carrier liquid and incubate the one or more of the encapsulated cellular spheroids such that the living cells multiply.

Some example implementations provide a method for producing an encapsulated cellular spheroid comprises containing, in a source liquid reservoir, a source liquid comprising a mixture of a living cell suspension and a liquid extracellular analog at a temperature less than a threshold temperature of the extracellular analog; containing, in a vertical solidification column, a carrier liquid at a temperature greater than the threshold temperature of the extracellular analog and having a greater density than a density of the source liquid; dispensing, by a source liquid injector in fluid communication with the source liquid reservoir and the vertical solidification column, droplets of the source liquid into a lower portion of the vertical solidification column, the greater density of the carrier liquid allowing the lesser dense source liquid droplets to travel from the lower portion of the vertical solidification column to an upper portion thereof and heat to the temperature greater than the threshold temperature of the extracellular analog, wherein the liquid extracellular analog of the source liquid droplets irreversibly semi-solidifies at the temperature and encapsulates the living cell suspension within, such that living cells in the encapsulated living cell suspension adhere to one another in a spherical mass and form the encapsulated cellular spheroid.

In some example implementations of the method of any preceding example implementation, or any combination of any preceding example implementations, containing the carrier liquid in the vertical solidification column comprises containing the carrier liquid in the vertical solidification column defining a height extending between the lower portion and the upper portion which corresponds to an amount of time that it takes for the dispensed source liquid droplets to heat to the temperature greater than the liquid-solid threshold temperature.

In some example implementations of the method of any preceding example implementation, or any combination of any preceding example implementations, dispensing droplets of the source liquid by the source liquid injector comprises dispensing droplets of the source liquid by the source liquid injector including an aperture through which the source liquid droplets are dispensed into the carrier liquid contained in the vertical solidification column, and wherein a volume of each of the dispensed source liquid droplets is determined by a size of the aperture, a flow rate of the source liquid through the source liquid injector, a surface tension of the source liquid, and a buoyancy of the source liquid droplets within the carrier fluid.

In some example implementations of the method of any preceding example implementation, or any combination of any preceding example implementations, the method further comprises stirring, using a source liquid stirring mechanism, the source liquid contained in the source liquid reservoir; maintaining, using a source liquid cooler, a temperature of the source liquid contained in the source liquid reservoir at a temperature less than the liquid-solid threshold temperature of the extracellular analog; and controlling, using a source liquid pump in fluid communication with the source liquid reservoir, a flow rate of the source liquid and directing the source liquid to the source liquid injector.

In some example implementations of the method of any preceding example implementation, or any combination of any preceding example implementations, the method further comprises directing the encapsulated cellular spheroid from the upper portion of the vertical solidification column into a flow of circulating carrier liquid using a flow guide in fluid communication with the vertical solidification column.

In some example implementations of the method of any preceding example implementation, or any combination of any preceding example implementations, the method further comprises a spheroid separator arranged after the flow guide and along a direction of the flow of circulating carrier liquid, and configured to separate the encapsulated cellular spheroid from the flow of circulating carrier liquid and direct the encapsulated cellular spheroid to a spheroid collector and direct the flow of circulating carrier liquid to a carrier liquid reservoir in fluid communication with the vertical solidification column.

In some example implementations of the method of any preceding example implementation, or any combination of any preceding example implementations, the method further comprises removing, using a carrier liquid filter arranged to receive the flow of circulating carrier liquid from the spheroid separator, residual semi-solid source liquid from the flow of circulating carrier liquid, and directing the flow of circulating carrier liquid to the carrier liquid reservoir; maintaining, using a carrier liquid heater, the circulating carrier liquid contained in the carrier liquid reservoir at a temperature greater than the liquid-solid threshold of the extracellular analog; and maintaining, using a carrier liquid circulating pump, the flow of circulating carrier liquid through the vertical solidification column.

In some example implementations of the method of any preceding example implementation, or any combination of any preceding example implementations, the method further comprises receiving, using a container of nutrient media, one or more of the encapsulated cellular spheroids separated by the spheroid separator from the flow of circulating carrier liquid and incubating the one or more of the encapsulated cellular spheroids therein such that the living cells multiply.

These and other features, aspects, and advantages of the present disclosure will be apparent from a reading of the following detailed description together with the accompanying figures, which are briefly described below. The present disclosure includes any combination of two, three, four, or more features or elements set forth in this disclosure or recited in any one or more of the claims, regardless of whether such features or elements are expressly combined or otherwise recited in a specific embodiment description or claim herein. This disclosure is intended to be read holistically such that any separable features or elements of the disclosure, in any of its aspects and embodiments, should be viewed as intended to be combinable, unless the context of the disclosure clearly dictates otherwise.

It will therefore be appreciated that this Brief Summary is provided merely for purposes of summarizing some example implementations so as to provide a basic understanding of some aspects of the disclosure. Accordingly, it will be appreciated that the above described example implementations are merely examples and should not be construed to narrow the scope or spirit of the disclosure in any way. Other example implementations, aspects and advantages will become apparent from the following detailed description taken in conjunction with the accompanying figures which illustrate, by way of example, the principles of some described example implementations.

### BRIEF DESCRIPTION OF THE FIGURES

Having thus described examples of the disclosure in general terms, reference will now be made to the accompanying figures, which are not necessarily drawn to scale, and wherein:

FIG. 1 illustrates an example implementation of an encapsulated cellular spheroid according to the present disclosure;

FIG. 2 illustrates an example implementation of a system for producing an encapsulated cellular spheroid according to the present disclosure; and

FIG. 3 illustrates an example implementation of a method for producing an encapsulated cellular spheroid according to the present disclosure.

### DETAILED DESCRIPTION

Some implementations of the present disclosure will now be described more fully hereinafter with reference to the accompanying figures, in which some, but not all implementations of the disclosure are shown. Indeed, various implementations of the disclosure may be embodied in many different forms and should not be construed as limited to the implementations set forth herein; rather, these example implementations are provided so that this disclosure will be thorough and complete, and will fully convey the scope of the disclosure to those skilled in the art. For example, unless specified otherwise or clear from context, references to first, second or the like should not be construed to imply a particular order. A feature may be described as being above another feature (unless specified otherwise or clear from context) may instead be below, and vice versa; and similarly, features described as being to the left of another feature else may instead be to the right, and vice versa. As used herein, unless specified otherwise or clear from context, the “or” of a set of operands is the “inclusive or” and thereby true if and only if one or more of the operands is true, as opposed to the “exclusive or” which is false when all of the operands are true. Thus, for example, “[A] or [B]” is true if [A] is true, or if [B] is true, or if both [A] and [B] are true. Further, the articles “a” and “an” mean “one or more,” unless specified otherwise or clear from context to be directed to a singular form. Like reference numerals refer to like elements throughout.

A system and method for producing an encapsulated cellular spheroid is disclosed herein. Specifically, a system and method for producing spheres of extracellular analog containing living cells, including cancer cells, non-cancerous cells, or combinations of dissimilar cells whether cancerous or non-cancerous, for use in cancer research experiments, or more generally research pertaining to living cells, is described herein. In practice, the produced encapsulated cellular spheroids may be used in cancer research experiments that rely upon an experimental environment that mimics the environment of an object (e.g., a living organism, such as a human or other mammalian body) where cancer cells live and grow within the microscopic structures of tissues and organic matrices.

In particular, the system and method described herein may be automated so as to produce a high-volume of encapsulated cellular spheroids, where traditional production of such spheroids is a tedious, time-consuming, and error-prone manual process. At the same time, example implementations of the present disclosure may enable more scientists at more research institutions to pursue new lines of research that were previously out of reach because of the inherent difficulty in producing suitable spheroids of living cancer cells.

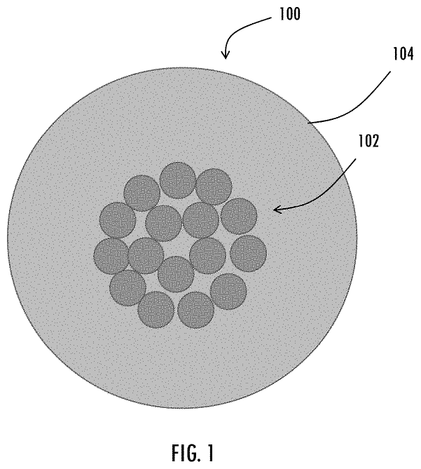

FIG. 1 illustrates an example implementation of an encapsulated cellular spheroid, generally referred to as 100, produced by the system and method disclosed herein. The encapsulated cellular spheroid 100 may be a three-dimensional spheroid comprising a spherical mass of living cells 102 colonizing or adhering to one another. The spherical mass of the living cells 102 may be a three-dimensional mass similar in shape to a spheroid, although other shapes, such as an ovoid are also contemplated herein. Such living cells may include, for example, cancer cells; although, other types of living cells are also contemplated by this disclosure.

The mass of living cells 102 may live within a matrix formed by an extracellular analog 104. The extracellular analog may be infused within and substantially surround (encapsulate) the mass of living cells 102. For example, the extracellular analog 104 may comprise macromolecules, which polymerize to form the matrix or a scaffold. The matrix or scaffold of the extracellular analog 104 provides a structured environment for growth and sustainment of the mass of living cells 102 when the mass of living cells 102 are infused or otherwise introduced to the extracellular analog 104. The extracellular analog 104 may be collagen, or any other material with similar properties, including, but not limited to commercially available MATRIGEL™. The use of collagen or another material like collagen mimics the in-vivo extracellular matrix of the object (e.g., a living organism). Further, various materials may be added to the extracellular analog to control density, porosity, gaseous exchange, nutrient absorption, and other aspects of the environment provided by the encapsulating extracellular analog 104.

The extracellular analog 104 may irreversibly semi-solidify at a temperature greater than a liquid-solid threshold temperature of the extracellular analog 104 so as to encapsulate the spherical mass of living cells 102 within and form the encapsulated cellular spheroid 100. The liquid-solid threshold temperature of the extracellular analog 104 is a temperature where the extracellular analog 104 will remain liquid so long as its temperature remains below the liquid-solid threshold temperature and will irreversibly solidify or semi-solidify when the its temperature rises above liquid-solid threshold temperature. This ensures that the encapsulated cellular spheroids are stable and can be handled individually, can be stored in suspension in various containers, and can be used as the subject of various experiments over a wide range of temperatures (without melting and dissolving as would be the case if the extracellular analog was not irreversibly solid or semi-solid). In some example implementations, the liquid-solid threshold temperature of the extracellular analog 104 may be between about 4° C. and 37° C.

The extracellular analog 104 promotes formation of the spherical shape of the mass of living cells 102. In particular, the described characteristics of the extracellular analog 104 (i.e., its spherical shape that irreversibly semi-solidifies at a temperature greater than the liquid-solid threshold temperature) enable the extracellular analog 104 to retain its shape without need for a surface coating or adherent surface, which may interfere with the proper exchange of nutrients and dissolved gases. Thus, when the mass of living cells 102 are infused or introduced into the spherical droplet of the extracellular analog 104, the extracellular analog 104 uniformly or substantially uniformly surrounds the mass of living cells 102 living within the matrix of the spherical extracellular analog 104, and thereby promotes formation of a spherical mass of the living cells 102. The extracellular analog 104 may totally surround the mass of living cells 102 so that the living cells are wholly encapsulated within the extracellular analog 104, or the extracellular analog substantially or mostly surrounds the mass of living cells 102 so that the living cells are substantially or mostly encapsulated within the extracellular analog 104. Regardless, the mass of living cells 102 may be contained within the spherical droplet of extracellular analog 104 without an adherent surface that provides, in its interior, a protein scaffold for formation of a spherical mass of living cells, where in other circumstances adherent surfaces tend to promote formation of an undesirable monolayer cell colony (as is the case when using traditional methods and systems for forming such types of cells).

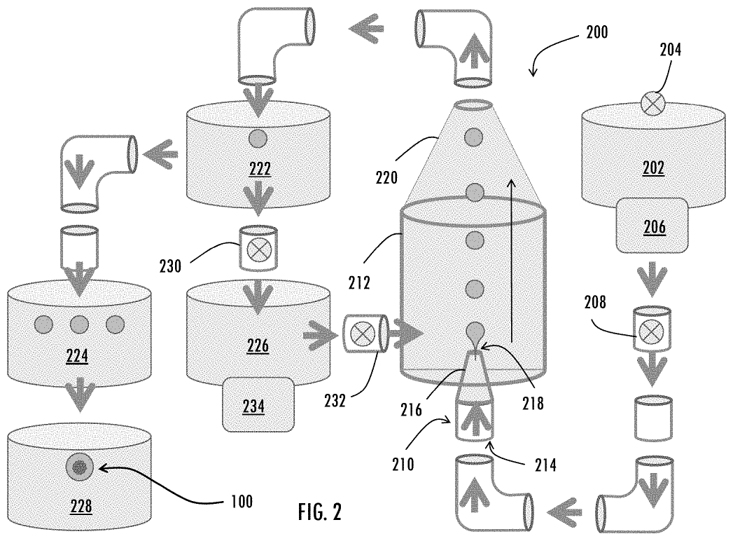

Turning now to FIG. 2, a system for producing an encapsulated cellular spheroid is disclosed, such system generally designated as reference numeral 200. The encapsulated cellular spheroid produced by the system 200 is, in some example implementations, an encapsulated cellular spheroid such as the encapsulated cellular spheroid 100, as described in reference to FIG. 1.

The system 200 may comprise a source liquid reservoir 202 containing a source liquid. The source liquid reservoir 202 may be a containment vessel that is able to store/contain a quantity of source liquid in a sterile environment. The source liquid may comprise a mixture of a living cell suspension and a liquid extracellular analog at a temperature less than a threshold temperature of the extracellular analog. The liquid extracellular analog may remain a liquid at temperatures less than or equal to a liquid-solid threshold temperature of the extracellular analog and may become irreversibly semi-solid when warmed to a temperature above the liquid-solid threshold of the extracellular analog. When contained within the source liquid reservoir 202, the source liquid may remain at a temperature less than or equal to the liquid-solid threshold temperature of the extracellular analog.

In some example implementations, the system 200 may further comprise a source liquid stirring mechanism 204 arranged to stir the source liquid contained in the source liquid reservoir 202. The source liquid stirring mechanism 204 may be arranged externally or internally relative to the source liquid reservoir 202. Such a mechanism may include, for example, a magnetic stirring device comprising a small coated magnet in the bottom portion of the source liquid reservoir that spins under the influence of a rotating magnetic field external to the source liquid reservoir. At certain time intervals, the source liquid stirring mechanism 204 may be actuated to circulate the source liquid contained within the source liquid reservoir 202 so as to maintain a uniform temperature of the source liquid. Otherwise, the source liquid stirring mechanism 204 constantly or continuously stirs the source liquid contained in the source liquid reservoir 202 at a rate sufficient to keep the living cells in suspense so as to enable a constant concentration of living cells in the source liquid delivered into the carrier liquid solidification column.

The system 200 may further comprise a source liquid cooler 206 arranged to maintain a temperature of the source liquid contained in the source liquid reservoir 202 at a temperature less than the liquid-solid threshold temperature of the extracellular analog. The source liquid cooler 206 may be arranged externally or internally relative to the source liquid reservoir 202. The source liquid cooler 206 may comprise, for example, a heat exchanger. The source liquid cooler 206 may be actuated when a temperature sensor in the source liquid reservoir 202 measures the temperature of the source liquid as heating, so that the source liquid is close to becoming hotter than the liquid-solid threshold temperature of the extracellular analog. At that point, the source liquid cooler 206 may be actuated to remove heat from source liquid contained in the source liquid reservoir 202.

A source liquid pump 208 may also be included in the system 200. The source liquid pump 208 may be in fluid communication with the source liquid reservoir 202 and may receive the source liquid from the source liquid reservoir 202 and control a flow rate of the source liquid from the source liquid reservoir 202. The source liquid may be directed to other components of the system 200 (i.e., a source liquid injector 210) at the flow rate defined by the source liquid pump 208. The source liquid pump 208 may be arranged in-line with the flow of the source liquid or external to the flow of the source liquid. In some example implementations, the source liquid pump 208 pressurizes the source liquid and directs pressurized source liquid to other components of the system 200.

The system 200 may further comprise a source liquid injector 210 in fluid communication with the source liquid reservoir 202 and a vertical solidification column 212 containing a carrier liquid at a temperature greater than the threshold temperature of the extracellular analog, and having a greater density than a density of the source liquid. The source liquid injector 210 may be arranged to dispense droplets of the source liquid into a lower portion of the vertical solidification column 212. The greater density of the carrier liquid may allow the lesser dense source liquid droplets to rise naturally and travel from the lower portion of the vertical solidification column 212 to an upper portion thereof, and heat to the temperature greater than the threshold temperature of the extracellular analog during travel from the lower portion to the upper portion of the vertical solidification column 212. Accordingly, pumps and other methods are thus not needed to force the droplets through the vertical solidification column 212.

The carrier liquid may be contained within the vertical solidification column 212 at a temperature greater than the liquid-solid threshold temperature of the extracellular analog, but not exceeding human body temperature. In particular, the carrier liquid may be heated to a predetermined temperature above the liquid solid threshold temperature of the extracellular analog so that the extracellular analog of the droplets of the source liquid can naturally polymerize without the side effects of chemically-induced polymerization.

Example carrier liquids may comprise a non-conductive, thermally and chemically stable, gas permeable, bio-inert substance such as FLUORINERT™ Liquid FC-40. Such example carrier liquids are biologically safe, where the semi-solid spheres of the extracellular analog infused within living cells can form without side effects resulting from chemical contact with the carrier liquid. The example carrier liquids are also hydrophobic so that the carrier liquid repels water and does not adhere to wet surfaces, and so that the semi-solid spheres of the extracellular analog infused within living cells can form without dispersing into the carrier liquid and so that the source liquid injected into the carrier liquid will form spherical droplets by virtue of the separation between the source liquid and the carrier liquid and the shape forming influence of the surface tension of the source liquid. The example carrier liquids are also gas-permeable so that the carrier liquid can be infused within an appropriate concentration of gases, whereby the living cells infused within the droplet of source liquid injected into the carrier liquid will have adequate gaseous exchange.

The source liquid injector 210 may comprise a source liquid inlet 214 at a first end which receives the source liquid at the flow rate defined by the source liquid pump 208. An injector tip 216 defining an injector aperture 218 may be at an opposing, second end of the source liquid injector 210 and through which the source liquid droplets are dispensed into the carrier liquid contained in the vertical solidification column 212. The injector tip 216 may be cooled to a temperature below the temperature of the source liquid, where the temperature of the source liquid is less than or equal to the liquid-solid threshold of the extracellular analog. This is done in order to prevent premature solidification of the source liquid while passing through the injector aperture 218. In order to further prevent solidification of the source liquid while passing through the injector aperture 218, an injector tip cover may be provided over the injector aperture 218 so as to prevent solidification of the source liquid on an external portion of the injector tip 216. The injector tip cover may be configured with a “non-stick”, temperature-insulating surface having properties that prevent agglomeration of the source liquid on the external portion of the injector tip 216 and/or within an internal portion of the injector tip 216 resulting from undesirable heat transfer from the warmer carrier liquid to the injector tip 216.

A volume of each of the source liquid droplets dispensed by the source liquid injector 206 may be determined by a number of system variables, including, but not limited to a size of the injector aperture 218, the flow rate of the source liquid through the source liquid injector 210 (as determined by the source liquid pump 208), a surface tension of the source liquid, and a buoyancy of the source liquid droplets within the carrier fluid as determined by a difference between a density of the source liquid and a density of the carrier liquid. Each of these system variables may be adjusted as necessary depending on the desired performance of the system 200. A rate at which the source liquid droplets are dispensed by the source liquid injector 206 may be controlled by a control mechanism (e.g., a non-transitory computer readable medium including a hardware processor and a memory) controlling the source liquid pump.

The vertical solidification column 212 may include a container that defines a width that allows the dispensed source liquid droplets to travel unimpeded (i.e., without contacting one another or a side of the container) from the lower portion to the upper portion, and a height extending between the lower portion and the upper portion, which corresponds to an amount of time that it takes for the dispensed source liquid droplets to heat to the temperature greater than the liquid-solid threshold temperature of the extracellular analog. In other words, as the dispensed source liquid droplets travel from the lower portion to the upper portion of the vertical solidification column 212, the dispensed source liquid droplets begin to heat to the temperature greater than the threshold temperature of the extracellular analog. The vertical solidification column 212 is structured so that by the time the dispensed source liquid droplets reach the upper portion of the vertical solidification column 212, the source liquid droplets have reached a temperature greater than the threshold temperature of the extracellular analog, and the liquid extracellular analog of the source liquid droplets has irreversibly semi-solidfied and thereby encapsulated the living cell suspension within. As such, the living cells in the encapsulated cell suspension adhere to one another in a spherical mass and form the encapsulated cellular spheroid 100.

In some example implementations, the system 200 includes a flow guide 220 in fluid communication with the vertical solidification column 212 and arranged to direct the encapsulated cellular spheroid(s) from the upper portion of the vertical solidification column 212 into a flow of circulating carrier liquid. The system 200 may also include a spheroid separator 222 arranged after the flow guide 220 and along a direction of the flow of circulating carrier liquid. The spheroid separator 222 may be configured to separate the encapsulated cellular spheroid from the flow of circulating carrier liquid and direct the encapsulated cellular spheroid to a spheroid collector 224 and direct the flow of circulating carrier liquid to a carrier liquid reservoir 226 in fluid communication with the vertical solidification column 212.

The spheroid separator 222 may be a centrifuge or other type of filtering mechanism that is able to separate encapsulated cellular spheroids of a predetermined volume along with a small quantity of the circulating carrier liquid from a remaining quantity of the circulating carrier liquid. The small quantity of the circulating carrier liquid may be enough such that the encapsulated cellular spheroids may be separated and transferred to a container of nutrient media for incubation. For example, the spheroid separator 222 is capable of separating the encapsulated spheroids with diameter about 1 mm or greater using a physical filter with pore size somewhat less than about 1 mm so as to allow carrier fluid and smaller ill-formed spheroids to pass through to the carrier liquid filter 230.

The spheroid collector 224 may be a vessel that is arranged to receive individual encapsulated cellular spheroids along with the small quantity of circulating carrier liquid. The spheroid collector 224 may only receive the encapsulated cellular spheroids of the predetermined diameter that the spheroid separator 222 has directed to the spheroid collector 224. For example, spheroids separated by the spheroid separator 222 may be of diameter at least about 1 mm and no greater than about 2 mm. Encapsulated cellular spheroids received in the spheroid collector 224 may be directed to a container of nutrient media 228, or may be otherwise preserved and shipped. This is beneficial as the encapsulated cellular spheroids (e.g., 100) can then be handled individually (e.g. transferred between container of nutrient media) without damaging the encapsulated cellular spheroid within.

The container of nutrient media 228 may be arranged to receive one or more of the encapsulated cellular spheroids separated by the spheroid separator 222 from the flow of circulating carrier liquid and incubate the one or more of the encapsulated cellular spheroids such that the living cells multiply. The spheroid separator 222 may direct the encapsulated cellular spheroids of the predetermined volume directly to the container of nutrient media 228 or may direct the encapsulated cellular spheroids first to the spheroid collector 224. Regardless, the container of nutrient media 228 may contain a material that incubates the encapsulated spheroids and allows the living cells to multiply, and where the internal spheroid of the living cells may grow to a size of about 200 μm or as much as about 750 μm after a period of incubation (e.g., 4 days). Further, a layer of agarose may be used to coat a bottom surface of the container of nutrient media 228.

The nutrient media may be a sustaining solution that provides nutrients and dissolved gases required by the living cells within the sphere of the extracellular analog (thereby reducing the manual labor required to successfully cultivate cellular spheroids). The nutrient media may have a density approximately equal to the density of the encapsulated cellular spheroids so as to avoid aggregation of the encapsulated cellular spheroids at a top or a bottom of the nutrient solution within in the container 228. The mass of living cells within the semi-solid spheres of extracellular analog achieve a spherical shape by virtue of that fact that semi-solid spheres of extracellular analog are suspended in or within a nutrient solution and the living cells will have limited or no contact with an adherent surface of the container holding the nutrient solution.

A carrier liquid filter 230 may be interposed between the carrier liquid reservoir 226 and the spheroid separator 220. The carrier liquid filter 230 may thus be arranged to receive the remaining flow of circulating carrier liquid from the spheroid separator 222, remove residual semi-solid source liquid from the flow of circulating carrier liquid, and direct the flow of circulating carrier liquid to the carrier liquid reservoir 226. The carrier liquid filter 230 may also filter out any encapsulated cellular spheroids that are smaller than the predetermined volume using, for example, a fine screened mesh, thereby assuring that the majority of the carrier liquid is free of debris. This filtration may also allow the flow of circulating carrier liquid to pass through to the carrier liquid reservoir 226. Such encapsulated cellular spheroids that are smaller than the predetermined volume may be discarded as waste material.

The carrier liquid reservoir 226 may be a container that is able to store or contain a predetermined quantity of the carrier liquid. Once the predetermined quantity of the carrier liquid is reached within the container, the carrier liquid reservoir may release the carrier liquid to the vertical solidification column 212. Otherwise, the carrier liquid reservoir 226 may direct a continuous stream of carrier liquid back to the vertical solidification column 212 at a flow rate that is controlled by a carrier liquid circulating pump 232. The carrier liquid circulating pump 232 is configured to maintain the flow of circulating carrier liquid through the vertical solidification column 212. The flow of carrier liquid recycled back to the vertical solidification column 212 is determined by a volume of carrier liquid remaining in the vertical solidification column 212 and a desired volume of carrier liquid to remain in the vertical solidification column 212. The flow rate of the recycled carrier liquid can thus be controlled depending on the volume needs of the vertical solidification column 212.

A carrier liquid heater 234 arranged to maintain the circulating carrier liquid contained in the carrier liquid reservoir 226 at a temperature greater than the liquid-solid threshold of the extracellular analog. The carrier liquid heater 234 may be arranged externally or internally relative to the carrier liquid reservoir 226. The carrier liquid heater 234 may comprise, for example, a heat exchanger. The carrier liquid heater 234 may be actuated when a temperature sensor in the carrier liquid reservoir 226 measures the temperature of the carrier liquid as cooling, so that the carrier liquid is close to becoming cooler than the liquid-solid threshold temperature of the extracellular analog. At that point, the carrier liquid heater 234 may be actuated to add heat to the carrier liquid contained in the carrier liquid reservoir 226 to maintain it at a temperature greater than the threshold temperature of the extracellular analog.

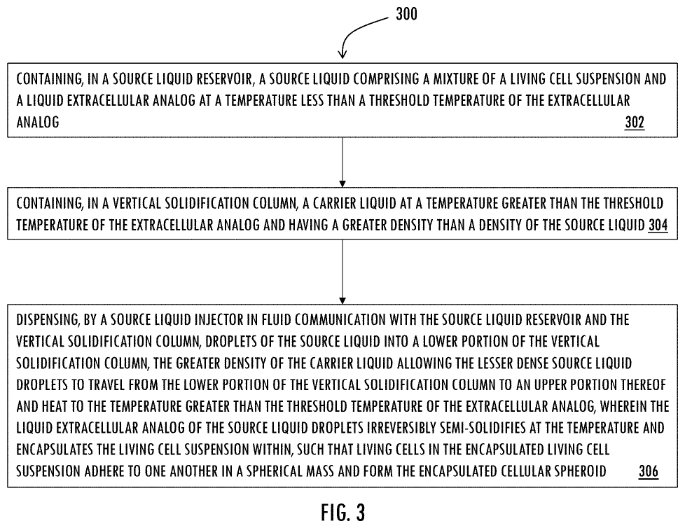

Referring now to FIG. 3, a method for producing an encapsulated cellular spheroid is disclosed, such method generally designated as reference numeral 300. The encapsulated cellular spheroid produced by the method 300 is, in some example implementations, an encapsulated cellular spheroid such as the encapsulated cellular spheroid 100, as described in reference to FIG. 1.

The method 300 may comprise a first step of containing, in a source liquid reservoir, a source liquid comprising a mixture of a living cell suspension and a liquid extracellular analog at a temperature less than a threshold temperature of the extracellular analog, 302. The method 300 may further comprise a second step of containing, in a vertical solidification column, a carrier liquid at a temperature greater than the threshold temperature of the extracellular analog and having a greater density than a density of the source liquid, 304. The method may still further comprise a third step of dispensing, by a source liquid injector in fluid communication with the source liquid reservoir and the vertical solidification column, droplets of the source liquid into a lower portion of the vertical solidification column, the greater density of the carrier liquid allowing the lesser dense source liquid droplets to travel from the lower portion of the vertical solidification column to an upper portion thereof and heat to the temperature greater than the threshold temperature of the extracellular analog, wherein the liquid extracellular analog of the source liquid droplets irreversibly semi-solidifies at the temperature and encapsulates the living cell suspension within, such that living cells in the encapsulated living cell suspension adhere to one another in a spherical mass and form the encapsulated cellular spheroid, 306.

Many modifications and other implementations of the disclosure set forth herein will come to mind to one skilled in the art to which this disclosure pertains having the benefit of the teachings presented in the foregoing descriptions and the associated figures. Therefore, it is to be understood that the disclosure is not to be limited to the specific implementations disclosed and that modifications and other implementations are intended to be included within the scope of the appended claims. Moreover, although the foregoing descriptions and the associated figures describe example implementations in the context of certain example combinations of elements and/or functions, it should be appreciated that different combinations of elements and/or functions may be provided by alternative implementations without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1. An encapsulated cellular spheroid comprising:
a spherical mass of living cells adhering to one another and living within a matrix formed by an extracellular analog;
the extracellular analog infused within and substantially surrounding the spherical mass of living cells and irreversibly semi-solidifying at a temperature greater than a liquid-solid threshold temperature of the extracellular analog so as to encapsulate the spherical mass of living cells within and form the encapsulated cellular spheroid.

2. The spheroid of claim 1, wherein the extracellular analog is collagen.

3. The spheroid of claim 1, wherein the living cells are cancer cells.

4. A system for producing an encapsulated cellular spheroid, the system comprising:
a source liquid reservoir containing a source liquid comprising a mixture of a living cell suspension and a liquid extracellular analog at a temperature less than a threshold temperature of the extracellular analog;
a vertical solidification column containing a carrier liquid at a temperature greater than the threshold temperature of the extracellular analog and having a greater density than a density of the source liquid; and
a source liquid injector in fluid communication with the source liquid reservoir and the vertical solidification column and being arranged to dispense droplets of the source liquid into a lower portion of the vertical solidification column, the greater density of the carrier liquid allowing the lesser dense source liquid droplets to travel from the lower portion of the vertical solidification column to an upper portion thereof and heat to the temperature greater than the threshold temperature of the extracellular analog,
wherein the liquid extracellular analog of the source liquid droplets irreversibly semi-solidifies at the temperature and encapsulates the living cell suspension within, such that living cells in the encapsulated living cell suspension adhere to one another in a spherical mass and form the encapsulated cellular spheroid.

5. The system of claim 4, wherein the vertical solidification column defines a height extending between the lower portion and the upper portion which corresponds to an amount of time that it takes for the dispensed source liquid droplets to heat to the temperature greater than the liquid-solid threshold temperature.

6. The system of claim 4, wherein the source liquid injector includes an aperture through which the source liquid droplets are dispensed into the carrier liquid contained in the vertical solidification column, and wherein a volume of each of the dispensed source liquid droplets is determined by a size of the aperture, a flow rate of the source liquid through the source liquid injector, a surface tension of the source liquid, and a buoyancy of the source liquid droplets within the carrier fluid.

7. The system of claim 4, further comprising:
a source liquid stirring mechanism arranged to stir the source liquid contained in the source liquid reservoir;
a source liquid cooler arranged to maintain a temperature of the source liquid contained in the source liquid reservoir at a temperature less than the liquid-solid threshold temperature of the extracellular analog; and
a source liquid pump in fluid communication with the source liquid reservoir that controls a flow rate of the source liquid and directs the source liquid to the source liquid injector.

8. The system of claim 4, further comprising a flow guide in fluid communication with the vertical solidification column and arranged to direct the encapsulated cellular spheroid from the upper portion of the vertical solidification column into a flow of circulating carrier liquid.

9. The system of claim 8, further comprising a spheroid separator arranged after the flow guide and along a direction of the flow of circulating carrier liquid, and configured to separate the encapsulated cellular spheroid from the flow of circulating carrier liquid and direct the encapsulated cellular spheroid to a spheroid collector and direct the flow of circulating carrier liquid to a carrier liquid reservoir in fluid communication with the vertical solidification column.

10. The system of claim 9, further comprising:
a carrier liquid filter arranged to receive the flow of circulating carrier liquid from the spheroid separator, remove residual semi-solid source liquid from the flow of circulating carrier liquid, and direct the flow of circulating carrier liquid to the carrier liquid reservoir;
a carrier liquid heater arranged to maintain the circulating carrier liquid contained in the carrier liquid reservoir at a temperature greater than the liquid-solid threshold of the extracellular analog; and
a carrier liquid circulating pump configured to maintain the flow of circulating carrier liquid through the vertical solidification column.

12. The system of claim 10, further comprising a container of nutrient media arranged to receive one or more of the encapsulated cellular spheroids separated by the spheroid separator from the flow of circulating carrier liquid and incubate the one or more of the encapsulated cellular spheroids such that the living cells multiply.

13. A method for producing an encapsulated cellular spheroid, the method comprising:
containing, in a source liquid reservoir, a source liquid comprising a mixture of a living cell suspension and a liquid extracellular analog at a temperature less than a threshold temperature of the extracellular analog;
containing, in a vertical solidification column, a carrier liquid at a temperature greater than the threshold temperature of the extracellular analog and having a greater density than a density of the source liquid;
dispensing, by a source liquid injector in fluid communication with the source liquid reservoir and the vertical solidification column, droplets of the source liquid into a lower portion of the vertical solidification column, the greater density of the carrier liquid allowing the lesser dense source liquid droplets to travel from the lower portion of the vertical solidification column to an upper portion thereof and heat to the temperature greater than the threshold temperature of the extracellular analog,
wherein the liquid extracellular analog of the source liquid droplets irreversibly semi-solidifies at the temperature and encapsulates the living cell suspension within, such that living cells in the encapsulated living cell suspension adhere to one another in a spherical mass and form the encapsulated cellular spheroid.

14. The method of claim 13, wherein containing the carrier liquid in the vertical solidification column comprises containing the carrier liquid in the vertical solidification column defining a height extending between the lower portion and the upper portion which corresponds to an amount of time that it takes for the dispensed source liquid droplets to heat to the temperature greater than the liquid-solid threshold temperature.

15. The method of claim 13, wherein dispensing droplets of the source liquid by the source liquid injector comprises dispensing droplets of the source liquid by the source liquid injector including an aperture through which the source liquid droplets are dispensed into the carrier liquid contained in the vertical solidification column, and wherein a volume of each of the dispensed source liquid droplets is determined by a size of the aperture, a flow rate of the source liquid through the source liquid injector, a surface tension of the source liquid, and a buoyancy of the source liquid droplets within the carrier fluid.

16. The method of claim 13, further comprising:
stirring, using a source liquid stirring mechanism, the source liquid contained in the source liquid reservoir;
maintaining, using a source liquid cooler, a temperature of the source liquid contained in the source liquid reservoir at a temperature less than the liquid-solid threshold temperature of the extracellular analog; and
controlling, using a source liquid pump in fluid communication with the source liquid reservoir, a flow rate of the source liquid and directing the source liquid to the source liquid injector.

17. The method of claim 13, further comprising directing the encapsulated cellular spheroid from the upper portion of the vertical solidification column into a flow of circulating carrier liquid using a flow guide in fluid communication with the vertical solidification column.

18. The method of claim 17, further comprising a spheroid separator arranged after the flow guide and along a direction of the flow of circulating carrier liquid, and configured to separate the encapsulated cellular spheroid from the flow of circulating carrier liquid and direct the encapsulated cellular spheroid to a spheroid collector and direct the flow of circulating carrier liquid to a carrier liquid reservoir in fluid communication with the vertical solidification column.

19. The method of claim 18, further comprising:
removing, using a carrier liquid filter arranged to receive the flow of circulating carrier liquid from the spheroid separator, residual semi-solid source liquid from the flow of circulating carrier liquid, and directing the flow of circulating carrier liquid to the carrier liquid reservoir;
maintaining, using a carrier liquid heater, the circulating carrier liquid contained in the carrier liquid reservoir at a temperature greater than the liquid-solid threshold of the extracellular analog; and
maintaining, using a carrier liquid circulating pump, the flow of circulating carrier liquid through the vertical solidification column.

20. The method of claim 19, further comprising receiving, using a container of nutrient media, one or more of the encapsulated cellular spheroids separated by the spheroid separator from the flow of circulating carrier liquid and incubating the one or more of the encapsulated cellular spheroids therein such that the living cells multiply.
